(19) United States
(12) Patent Application Publication
(10) Pub. No.: US 2025/0165662 A1
(43) Pub. Date: May 22, 2025

(54) FORGING FLAW DETERMINATION SYSTEM, FORGING FLAW DETERMINATION DEVICE, AND FORGING FLAW DETERMINATION METHOD

(71) Applicants: TOYOTA PRODUCTION ENGINEERING, Munakata City (JP); TOYOTA JIDOSHA KABUSHIKI KAISHA, Toyota-shi (JP)

(72) Inventors: Moe OISHI, Seto-shi (JP); Kazuhiro SUZUKI, Miyoshi-shi (JP); Katsuya YOSHIDA, Toyota-shi (JP); Kunihito ONA, Okazaki-shi (JP)

(73) Assignees: TOYOTA PRODUCTION ENGINEERING, Munakata City (JP); TOYOTA JIDOSHA KABUSHIKI KAISHA, Toyota-shi (JP)

(21) Appl. No.: 18/938,552

(22) Filed: Nov. 6, 2024

(30) Foreign Application Priority Data
Nov. 17, 2023 (JP) 2023-196160

(51) Int. Cl. G06F 30/17; G06F 30/23; G06F 119/18

(52) U.S. Cl. CPC G06F 30/17; G06F 30/23; G06F 2119/18

(57) ABSTRACT

A forging flaw determination system includes a forging analysis device and a forging flaw determination device. The forging analysis device is configured to generate a forged article model in a plurality of forging processes for forging, and includes a first controller. The first controller is configured to calculate a strain rate based on an analysis mesh for the forged article model. The forging flaw determination device is configured to predict, based on the forged article model, the presence or absence of a flaw phenomenon when a forged article is formed in each forging process for the forging, and includes a second controller. The second controller is configured to determine the presence or absence of the flaw phenomenon based on the strain rate and a face angle between faces of adjacent analysis meshes.

### CROSS-REFERENCE TO RELATED APPLICATION

The present application claims priority to Japanese Patent Application No. 2023-196160 filed on Nov. 17, 2023. The disclosure of the above-identified application, including the specification, drawings, and claims, is incorporated by reference herein in its entirety.

### BACKGROUND

### 1. Technical Field

The present disclosure relates to a forging flaw determination system, a forging flaw determination device, and a forging flaw determination method.

### 2. Description of Related Art

Forging is often performed by hitting an ingot or cylindrical metal block with a hammer or die to apply large force to the ingot or the block and plastically deform the ingot or the block into a shape. During such forging, a raw material may come together during die-sinking of the die, which causes closed flaws.

For this reason, a technique for predicting in advance a place where a flaw will occur during forging by using an analysis technique is known. For example, Japanese Unexamined Patent Application Publication No. 2022-035154 (JP 2022-035154) discloses a defect risk assessment method using a numerical simulation of a forging process based on a finite element method (see, for example, JP 2022-035154).

### SUMMARY

However, in a case where an analysis technique typified by JP 2022-035154 is used, when reconstruction of a mesh caused by plastic deformation occurs, the shape of the flaw becomes invisible. Therefore, in order to find a flaw, it is necessary to observe a forging process sequentially, which causes a problem of requiring human labor. In particular, there is also a problem that determination on the timings and locations at which flaws occur depends on experiences of workers, which restricts the workers.

The present disclosure provides a forging flaw determination system, a forging flaw determination device, and a forging flaw determination method that can efficiently determine occurrence or non-occurrence of a flaw phenomenon during forging.

A forging flaw determination system according to a first aspect of the present disclosure includes a forging analysis device, and a forging flaw determination device. The forging analysis device is configured to generate a forged article model in a plurality of forging processes for forging and includes a first controller. The first controller is configured to calculate a strain rate based on an analysis mesh for the forged article model. The forging flaw determination device is configured to predict, based on the forged article model, presence or absence of a flaw phenomenon when a forged article is formed in each forging process for the forging and includes a second controller. The second controller is configured to determine the presence or absence of the flaw phenomenon based on the strain rate and a face angle between faces of adjacent analysis meshes.

Furthermore, in the forging flaw determination system according to the first aspect of the present disclosure, the first controller may be configured to generate the analysis mesh for the forged article model, may be configured to calculate a distance between the forged article model and a die, and may be configured to calculate the strain rate based on an amount of change in the forged article model between forging processes.

Furthermore, in the forging flaw determination system according to the first aspect of the present disclosure, the second controller may be configured to calculate the face angle between the faces of the adjacent analysis meshes, may be configured to calculate a predicted value of the flaw phenomenon based on the strain rate and the face angle, and may be configured to determine the presence or absence of the flaw phenomenon based on the predicted value and the distance.

Furthermore, in the forging flaw determination system according to the first aspect of the present disclosure, the second controller may be configured to calculate the predicted value of the flaw phenomenon by logistic regression analysis in which the strain rate and the face angle are set as explanatory variables and occurrence of the flaw phenomenon is set as an objective variable.

A forging flaw determination device according to a second aspect of the present disclosure is configured to predict, based on a forged article model in a plurality of forging processes for forging, presence or absence of a flaw phenomenon when a forged article is formed in each forging process for the forging. The forging flaw determination device includes a second controller. The second controller is configured to calculate a strain rate based on an analysis mesh for the forged article model, and configured to determine the presence or absence of the flaw phenomenon based on the strain rate and a face angle between faces of adjacent analysis meshes.

A forging flaw determination method according to a third aspect of the present disclosure is executed in a forging flaw determination system. The forging flaw determination system includes a forging analysis device configured to generate a forged article model in a plurality of forging processes for forging, and a forging flaw determination device configured to predict, based on the forged article model, presence or absence of a flaw phenomenon when a forged article is formed in each forging process for the forging. The forging flaw determination method includes calculating, by the forging analysis device, a strain rate based on an analysis mesh for the forged article model, and determining, by the forging flaw determination device, the presence or absence of the flaw phenomenon based on the strain rate and a face angle between faces of adjacent analysis meshes.

According to the present disclosure, the presence or absence of a flaw in forging can be efficiently determined.

### BRIEF DESCRIPTION OF THE DRAWINGS

Features, advantages, and technical and industrial significance of exemplary embodiments of the present disclosure will be described below with reference to the accompanying drawings, in which like signs denote like elements, and wherein:

FIG. 1A is a diagram showing an overview of a forging flaw determination system according to an embodiment;

FIG. 1B is a diagram showing an overview of the forging flaw determination system according to an embodiment;

FIG. 2 is a functional block diagram showing a configuration of a forging analysis device shown in FIG. 1B;

FIG. 3 is a diagram showing an example of generation of an analysis mesh;

FIG. 4 is a diagram showing an example of a calculation result of the distance between a forged article model and a die;

FIG. 5 is a diagram showing an example of a calculation result of a strain rate;

FIG. 6 is a functional block diagram showing a configuration of a forging flaw determination device shown in FIG. 1B;

FIG. 7 is a diagram showing an example of forging data shown in FIG. 6;

FIG. 8 is an explanatory diagram showing calculation of a threshold value;

FIG. 9 is an explanatory diagram showing calculation of a face angle;

FIG. 10 is a diagram showing an example of a calculation result of the face angle;

FIG. 11 is a flowchart showing a processing procedure of the forging analysis device shown in FIG. 1B;

FIG. 12 is a flowchart showing a processing procedure of the forging flaw determination device shown in FIG. 1B; and

FIG. 13 is a diagram showing a display example of the presence or absence of a flaw by the forging flaw determination device.

### DETAILED DESCRIPTION OF EMBODIMENTS

Embodiments of a forging flaw determination system, a forging flaw determination device, and a forging flaw determination method according to the present disclosure will be described hereunder in detail with reference to the drawings.

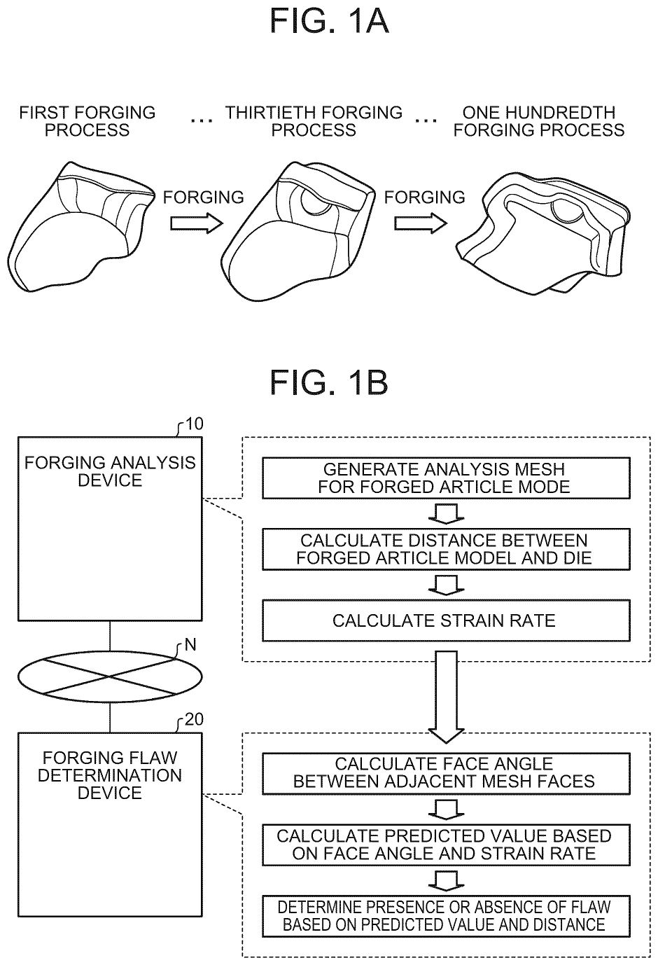

An overview of the forging flaw determination system according to the present embodiment will be described below. FIGS. 1A, 1B are explanatory diagrams showing an overview of the forging flaw determination system according to the embodiment.

### Overview of Forging Flaw Determination System

As shown in FIG. 1A, a product manufacturing process using a forging technique forms a product through a plurality of forging processes. Here, a forged article is prepared in a forging process 1 (first forging process), and forging is repeated at each site in each forging process. Forging passes through a forging process 30 (thirtieth forging process), and is completed in a forging process 100 (100th forging process). A shape that has been generated while a raw material comes together and closes inside a die used for forging during forging is called a “flaw,” and generally, analysis is used to predict in advance a place where the flaw will occur.

However, in analysis, when reconstruction of a mesh to be used for analysis occurs due to plastic deformation, the shape of the flaw becomes invisible, so that it is necessary to observe the forming process sequentially in order to find the flaw.

As shown in FIG. 1B, the present disclosure calculates a predicted value of a flaw based on a forged article model in each forging step, and determines the presence or absence of a flaw. The forging flaw determination system includes a forging analysis device 10, and a forging flaw determination device 20, which are connected to each other via a network N. The forging analysis device 10 generates a mesh for analyzing a forged article model. Thereafter, the forging analysis device 10 calculates the distance between the forged article model and the die. The forging analysis device 10 then calculates a strain rate in each analysis mesh. Here, the strain rate is a value obtained by dividing the amount of deformation per unit time by an original length.

Thereafter, the forging flaw determination device 20 acquires mesh data, distance data, and strain rate data from the forging analysis device 10. The forging flaw determination device 20 calculates the face angles of all adjacent mesh faces based on the mesh data. The forging flaw determination device 20 then calculates a predicted value based on the face angle and the strain rate, and determines the presence or absence of a flaw based on the predicted value and the distance. Here, the predicted value is a numerical value calculated using a prediction formula for logistic regression analysis in which an objective function is set to the presence or absence of a flaw and an explanatory function is set to the face angle and strain rate, which will be described in detail later.

### Configuration of Forging Analysis Device 10

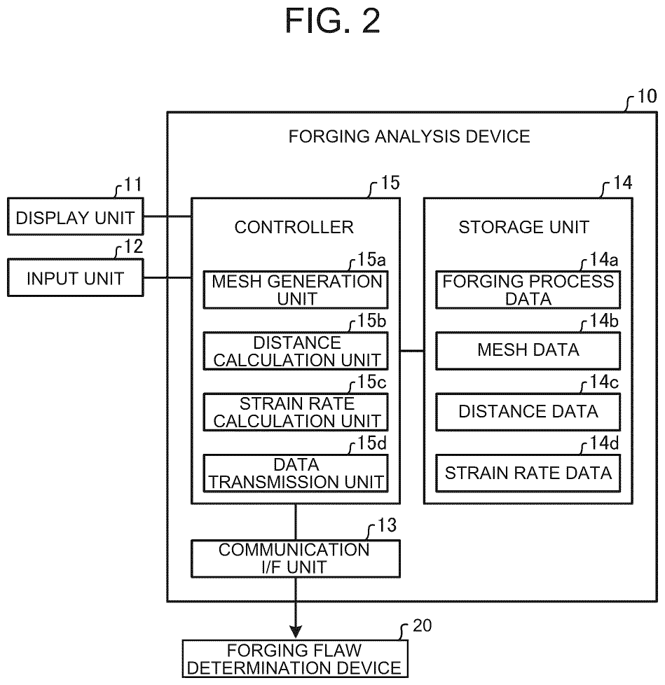

Next, the configuration of the forging analysis device 10 shown in FIG. 1B will be described. FIG. 2 is a functional block diagram showing the configuration of the forging analysis device 10 shown in FIG. 1B. As shown in FIG. 2, the forging analysis device 10 includes a display unit 11, an input unit 12, a communication I/F unit 13, a storage unit 14, and a controller 15. The display unit 11 is a display device such as a liquid crystal display that displays various information. The input unit 12 is an input device such as a mouse or keyboard. The communication T/F unit 13 is a communication interface unit for communicating with the forging flaw determination device 20 via the network N.

The storage unit 14 is a storage device such as a hard disk device or a non-volatile memory, and stores forging process data 14a, mesh data 14b, distance data 14c, and strain rate data 14d. The forging process data 14a is data of a forged article model in each forging process using a forging technique. The mesh data 14b is data of a mesh generated for the surface of the forged article model in order to analyze the forged article model. The distance data 14c is data of the distance between the die used for forging and the forged article model. The strain rate data 14d is data of the strain rate calculated in the analysis mesh.

The controller 15 is a controller for controlling the whole of the forging analysis device 10, and includes a mesh generation unit 15a, a distance calculation unit 15b, a strain rate calculation unit 15c, and a data transmission unit 15d. In practice, the processes corresponding to the mesh generation unit 15a, the distance calculation unit 15b, the strain rate calculation unit 15c, and the data transmission unit 15d are executed by loading programs for these units into a CPU and executing the programs. The controller 15 is an example of a first controller in the present disclosure.

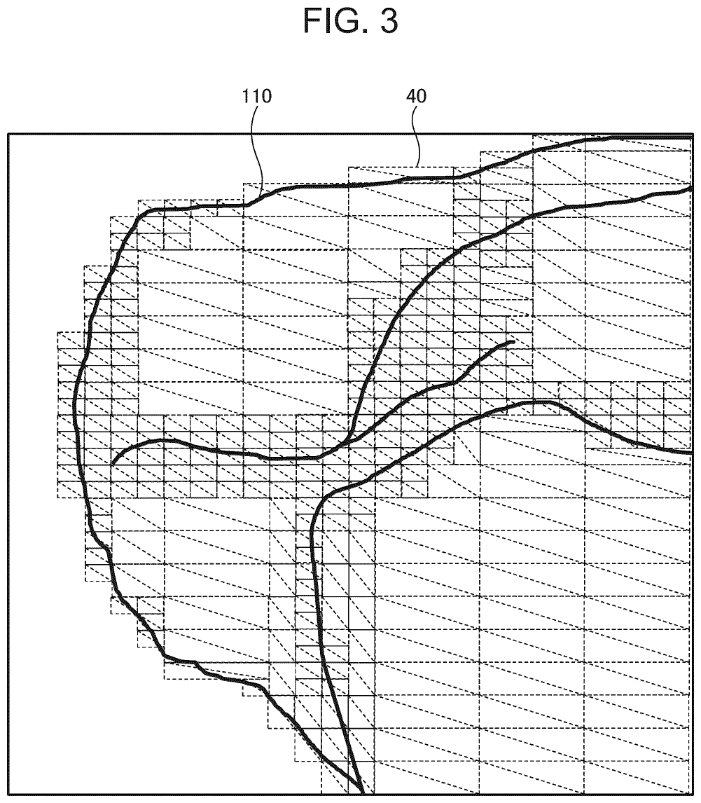

The mesh generation unit 15a is a processing unit for generating a mesh for analysis for the forged article model. With respect to the size of the mesh to be generated, the coarseness and fineness of the mesh is changed according to the shape of the forged article model. With respect to the size of the mesh, a dense mesh is generated in a portion where the shape of the forged article model changes, and a coarse mesh is generated in a portion where the shape of the forged article model does not change much (see FIG. 3). The generated mesh data is stored as mesh data 14b in the storage unit 14 in association with a forging process ID. Here, a case where a triangular mesh is generated using a diagonal of a rectangle will be described, however, any triangular mesh may be generated. The shape of the mesh may be a square or a hexagon.

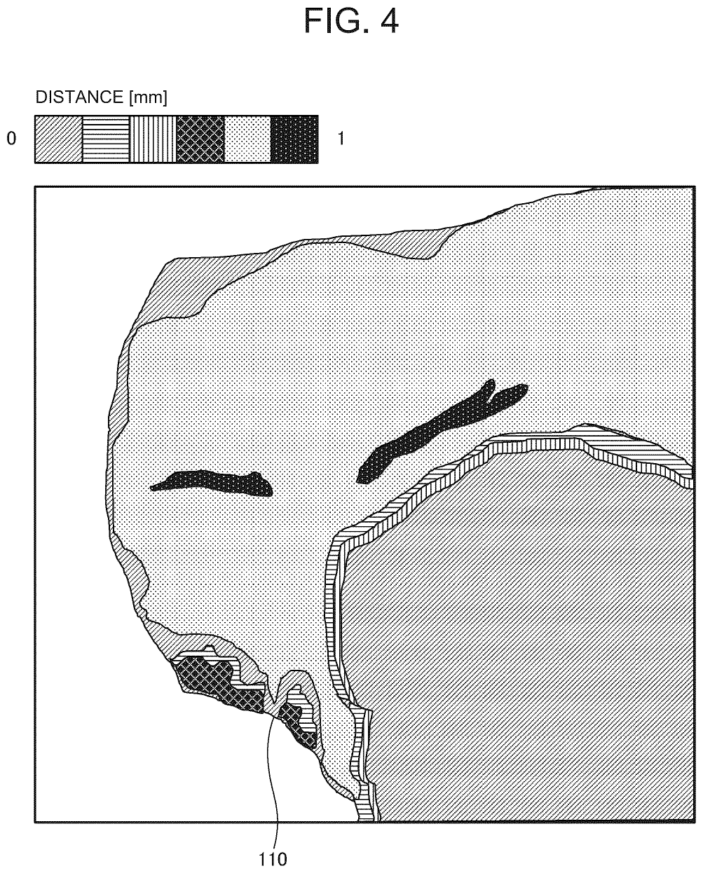

The distance calculation unit 15b is a processing unit for calculating the distance between the forged article model and the die to be used in each forging process for the forging. Specifically, as shown in FIG. 4, the distance calculation unit 15b calculates the distance between the forged article model 110 and the die (not shown) according to the forging process. When the distance is close to 0, it indicates a state in which the forged article model 110 and the die are close to each other, and when the distance is close to 1, it indicates a state where that the forged article model 110 and the die are located to be far away from each other. Although not shown, when the distance is a negative value, it indicates a state where the forged article model 110 and the die are in contact with each other. The calculated distance data is stored in the storage unit 14 as distance data 14c in association with the forging process ID.

The strain rate calculation unit 15c is a processing unit for calculating the strain rate in each mesh. The strain rate is a numerical value obtained by dividing the deformation amount per unit time of each mesh by an original length. Specifically, the strain rate of the forging process that generated the mesh is calculated while taking into account the deformation of the forging process that generated the mesh while inheriting a previous value until that point.

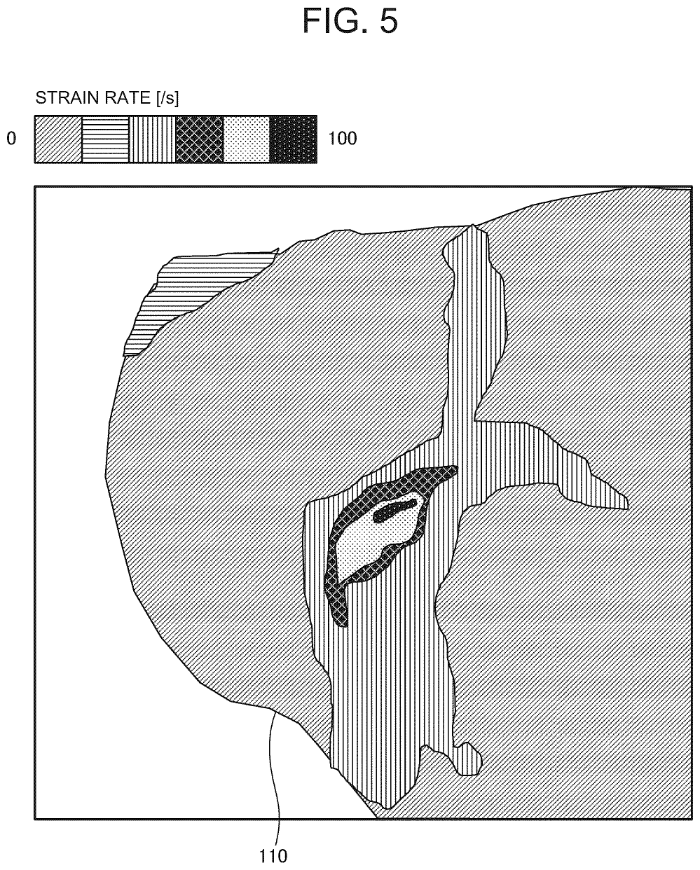

For example, as shown in FIG. 5, the strain rate calculation unit 15c calculates the strain rate corresponding to each mesh for the forged article model 110. With respect to the strain rate, in the forging process, a large strain rate is calculated for a portion where the shape of the forged article model 110 has changed, and a small strain rate is calculated for a portion where the shape of the forged article model 110 has little changed. The calculated strain rate is stored as strain rate data 14d in the storage unit 14 in association with the forging process ID.

The data transmission unit 15d is a processing unit for transmitting the mesh data 14b, the distance data 14c, and the strain rate data 14d to the forging flaw determination device 20 via the communication I/F unit 13.

### Configuration of Forging Flaw Determination Device 20

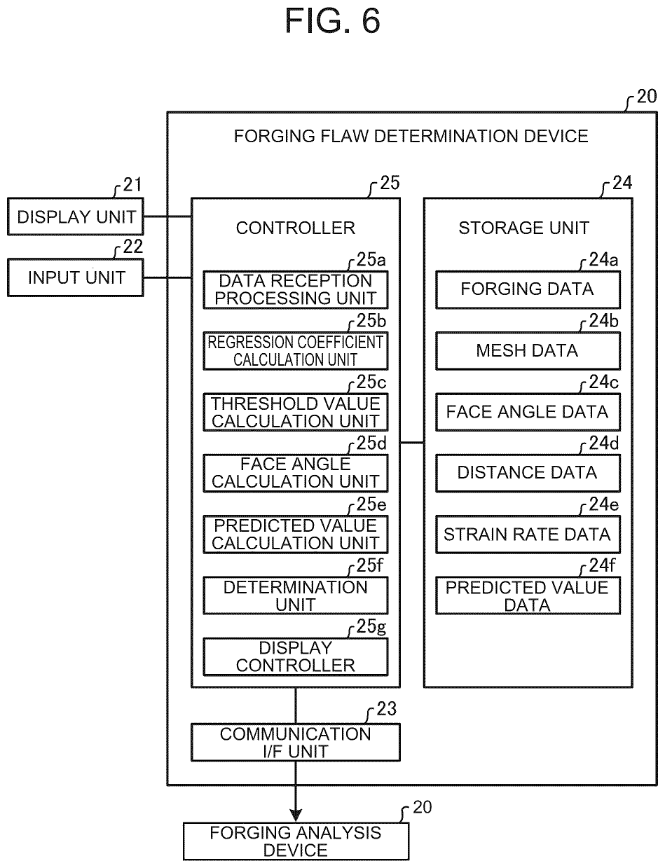

Next, the configuration of the forging flaw determination device 20 shown in FIG. 1B will be described. FIG. 6 is a functional block diagram showing the configuration of the forging flaw determination device 20 shown in FIG. 1B. As shown in FIG. 6, the forging flaw determination device 20 includes a display unit 21, an input unit 22, a communication I/F unit 23, a storage unit 24, and a controller 25. The display unit 21 is a display device such as a liquid crystal display for displaying various information. The input unit 22 is an input device such as a mouse or keyboard. The communication I/F unit 23 is a communication interface unit for communicating with the forging analysis device 10 via the network N.

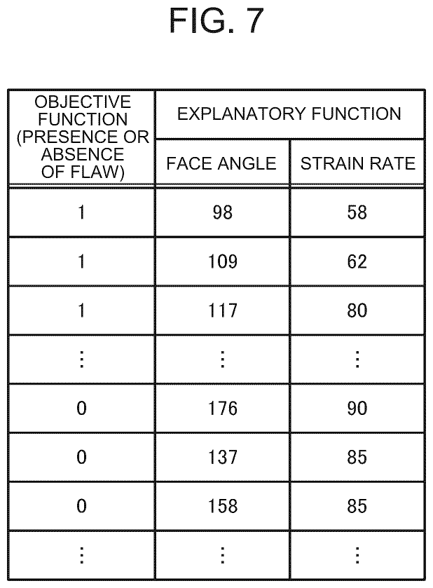

The storage unit 24 is a storage device such as a hard disk device or non-volatile memory, and stores forging data 24a, mesh data 24b, face angle data 24c, distance data 24d, strain rate data 24e, and predicted value data 24f. The forging data 24a is data on the presence or absence of a flaw which was formed on a forged article obtained by actually performing forging in advance, and face angles and strain rates obtained by analyzing portions where flaws occurred, such as data shown in FIG. 7.

Here, the presence or absence of a flaw “1” is associated with a face angle of “98” and a strain rate of “58”, the presence or absence of a flaw “1” is associated with a face angle of “109” and a strain rate of “62”, and the presence or absence of a flaw “1” is associated with a face angle of “117” and a strain rate of “80”.

Furthermore, the presence or absence of a flaw “0” is associated with a face angle of “176” and a strain rate of “90”, the presence or absence of a flaw “0” is associated with a face angle of “137” and a strain rate of “85”, and the presence or absence of a flaw “0” associated with a face angle of “158” and a strain rate of “85”.

The mesh data 24b is data of a mesh generated for the surface of the forged article model which is received to analyze the forged article model from the forging analysis device 10. The face angle data 24c is data of the intersection angle between the faces of adjacent meshes. The distance data 24d is data of the distance between the die used for forging and the forged article model which is received from the forging analysis device 10. The strain rate data 24e is data of the strain rate calculated in the analysis mesh which is received from the forging analysis device 10. The predicted value data 24f is numerical data to be used to determine the occurrence or non-occurrence of a flaw, which is calculated from a prediction formula calculated by logistic regression analysis based on the face angle data 24c and the strain rate data 24e.

The controller 25 is a controller for controlling the whole of the forging flaw determination device 20, and includes a data reception processing unit 25a, a regression coefficient calculation unit 25b, a threshold value calculation unit 25c, a face angle calculation unit 25d, a predicted value calculation unit 25e, a determination unit 25f, and a display controller 25g. In practice, programs for these units are loaded into a CPU and executed, thereby causing the data reception processing unit 25a, the regression coefficient calculation unit 25b, the threshold value calculation unit 25c, the face angle calculation unit 25d, the predicted value calculation unit 25e, the determination unit 25f, and the display controller 25g to execute processes corresponding to these units, respectively. The controller 25 is an example of a second controller in the present disclosure.

The data reception processing unit 25a is a processing unit for receiving data from the forging analysis device 10, and storing the data into the storage unit 24. Specifically, the data reception processing unit 25a receives the mesh data 14b from the forging analysis device 10, stores the mesh data 14b as the mesh data 24b into the storage unit 24, receives the distance data 14c from the forging analysis device 10, stores the distance data 14c as distance data 24d into the storage unit 24, receives the strain rate data 14d from the forging analysis device 10, and stores the strain rate data 14d as the strain rate data 24e into the storage unit 24.

The regression coefficient calculation unit 25b is a processing unit for calculating a regression coefficient representing the relation between an objective variable and an explanatory variable in a logistic regression analysis for calculating a predicted value for determining the presence or absence of a flaw in a forged article model using forging. Specifically, in this case, an objective variable is set to the presence or absence of a flaw, and explanatory variables are set to the mesh face angle and the strain rate.

Here, when the probability that binary data Y composed of I/O representing the presence or absence of a flaw as the objective variable is equal to 1 is represented by p(Y=1), the two explanatory variables of the face angle and the strain rate are represented by 1, x2 respectively, and regression coefficients are represented by β0, β1, β2 respectively, a prediction formula for logistic regression analysis can be expressed by Formula 1.

$$log ( \frac{p ( Y = 1 )}{1 - p ( Y = 1 )} ) = {β}_{0} + {β}_{1} {x}_{1} + {β}_{2} {x}_{2} ) \qquad ( 1 )$$

The log odds (logit) when the probability that the binary data Y composed of I/O representing the presence or absence of a flaw satisfies Y=1 is represented by P=p(Y=1) can be expressed as logit(P)=log(P/(1−P)).

The regression coefficient calculation unit 25b calculates the regression coefficient using the maximum likelihood method. The maximum likelihood method is a method for estimating parameters that maximize the likelihood from a given observation point. Specifically, the probability that the binary data Y satisfies Y=1 is represented by P=p(Y=1), and the probability that the binary data Y satisfies Y=0 is represented by 1−P=p(Y=0). Then, when n pieces of data Y (Y1, Y2, . . . , Yn) are independent of each other and p(Yi−1)=Pi is satisfied, the regression coefficient βi is calculated using the log-likelihood function such that the log-likelihood function is maximized. A Newton-Raphson method is used for this calculation.

The threshold value calculation unit 25c is a processing unit for calculating a threshold value to be used to determine the presence or absence of a flaw. Specifically, the predicted value is calculated based on the prediction formula based on logistic regression analysis and the forging data 24a using the regression coefficient calculated by the regression coefficient calculation unit 25b, and a numerical value between the minimum prediction value of the predicted value in a case where there is a flaw and the maximum prediction value of the predicted value in a case where there is no flaw is set as a threshold value.

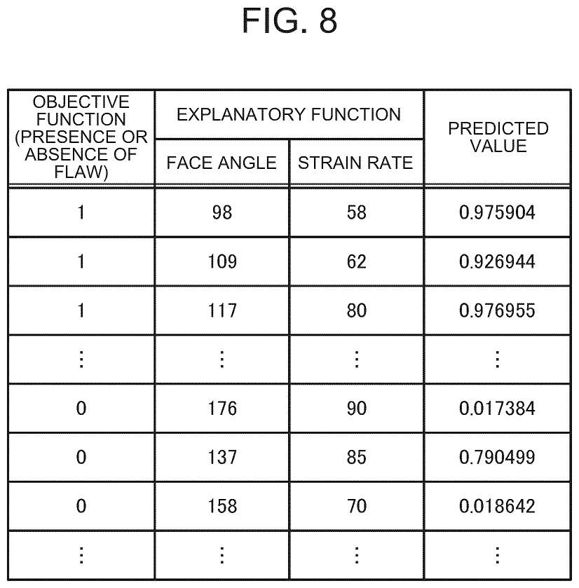

For example, as shown in FIG. 8, predicted values are calculated based on face angles and strain rates in a case where there is a flaw (objective function=1) and in a case where there is no flaw (objective function=0). Here, a predicted value of “0.975904” is associated with the objective function “1”, the face angle “98”, and the strain rate “58”, a predicted value of “0.926944” is associated with the objective function “1”, the face angle “109”, and the strain rate “62”, and a predicted value of “0.976955” is associated with the objective function “1”, the face angle “117”, and the strain rate “80”.

Furthermore, a predicted value of “0.017384” is associated with the objective function “0”, the face angle “176”, and the strain rate “90”, a predicted value of “0.790499” is associated with the objective function “0”, the face angle “137”, and the strain rate “85”, and a predicted value of “0.018642” is associated with the objective function “0”, the face angle “158”, and the strain rate “70”.

The threshold value calculation unit 25c sets, as a threshold value, a numerical value between the minimum value “0.926944” of the predicted value for the objective function “1” and the maximum value “0.790499” of the predicted value for the objective function “0”.

The face angle calculation unit 25d is a processing unit for reading out the mesh data 24b, calculating the angle between faces forming adjacent meshes for the meshes of the read-out mesh data 24b. The face angle calculation unit 25d calculates the face angles of all the adjacent meshes generated in the forged article model. The calculated angles of the faces forming the adjacent meshes are stored as the face angle data 24c into the storage unit 24 in association with the forging process ID.

The predicted value calculation unit 25e is a processing unit for calculating the predicted value using the prediction formula for logistic regression analysis based on the face angle data 24c and the strain rate data 24e. Specifically, the predicted value calculation unit 25e calculates the predicted value by reading out the face angle data 24c and the strain rate data 24e from the storage unit 24, and substituting them into the prediction formula based on the logistic regression analysis using the regression coefficient calculated by the regression coefficient calculation unit 25b. The predicted value is stored as the predicted value data 24f into the storage unit 24 in association with the forging process ID.

The determination unit 25f is a processing unit for determining the presence or absence of a flaw in a forged article model based on the distance data 24d and the predicted value data 24f. Specifically, the determination unit 25f reads out the distance data 24d and the predicted value data 24f from the storage unit 24, and in a case where the distance data 24d is a positive value, the determination unit 25f determines that there is a “flaw” when the predicted value data 24f is greater than a threshold value, and determines that there is “no flaw” when the predicted value data 24f is equal to or less than the threshold value. Furthermore, in a case where the distance data 24d is a negative value, the determination is skipped.

The display controller 25g is a processing unit for controlling display of the presence or absence of a flaw on a predetermined display unit 21 based on a result of the determination unit 25f.

### Angle Between Faces Forming Adjacent Meshes

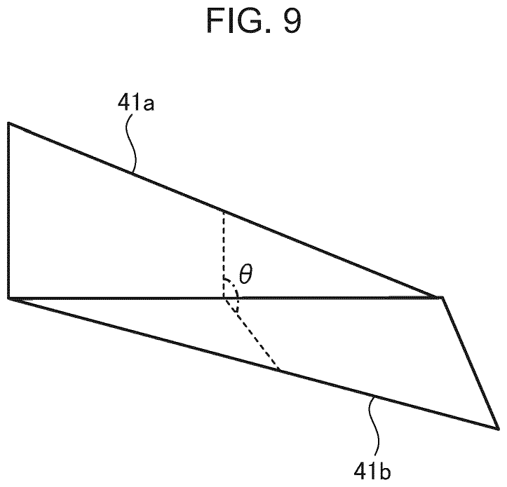

Next, the calculation of the angle (face angle) between the faces forming adjacent meshes in the face angle calculation unit 25d of the forging flaw determination device 20 will be described. FIG. 9 is an explanatory diagram showing the calculation of the face angle. As shown in FIG. 9, the face angle is defined as an intersection angle θ between a first mesh face 41a and a second mesh face 41b.

An equation of a plane for the first mesh face 41a can be expressed as a1x+b1y+c1z+d1=0. Furthermore, an equation of a plane for the second mesh face 41b can be expressed as a2x+b2y+c2z+d2=0. The intersection angle θ between the first mesh face 41a and the second mesh face 41b can be calculated using an equation (2).

$$θ = - 1 \times {cos}^{- 1} ( - 1 \times \frac{\vert {a}_{1} {a}_{2} + {b}_{1} {b}_{2} + {c}_{1} {c}_{2} \vert}{\sqrt{( {a}_{1}^{2} + {b}_{1}^{2} + {c}_{1}^{2} )} \times \sqrt{( {a}_{2}^{2} + {b}_{2}^{2} + {c}_{2}^{2} )}} ) ) \qquad ( 2 )$$

### Example of Calculation Result of Face Angle

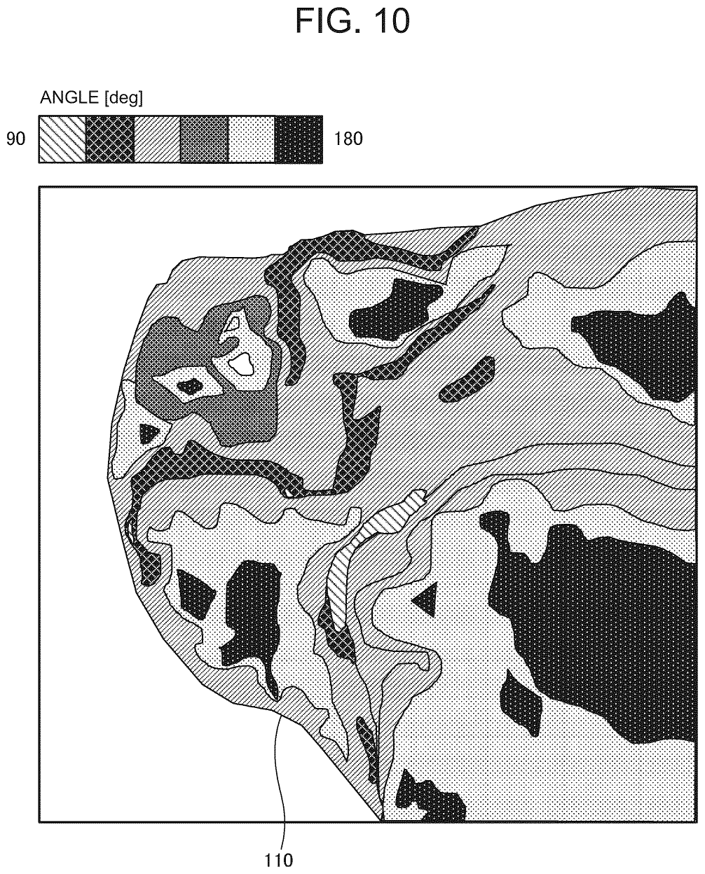

Next, an example of a face angle calculation result will be described. FIG. 10 is a diagram showing an example of a face angle calculation result. As shown in FIG. 10, the face angle calculation unit 25d calculates all angles of faces forming adjacent meshes generated in the forged article model 110. In a portion where the shape of the forged article model 110 changes little, the face angle indicates a value close to 180 degrees, and in a portion where the shape of the forged article model 110 changes greatly, the face angle indicates a value close to 90 degrees.

### Processing Procedure of Forging Analysis Device 10

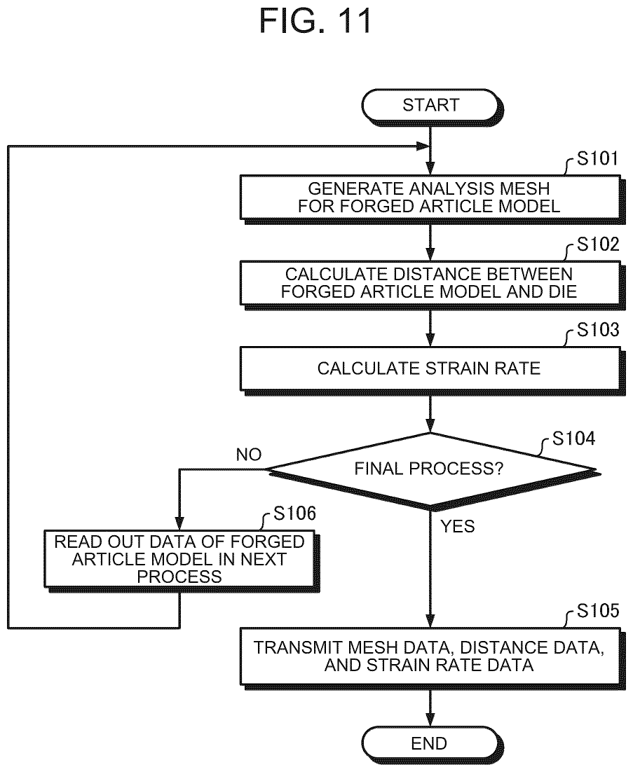

Next, a processing procedure of the forging analysis device 10 shown in FIG. 1B will be described. FIG. 11 is a flowchart showing the processing procedure of the forging analysis device 10 shown in FIG. 1B. As shown in FIG. 11, the forging analysis device 10 generates a mesh for analyzing a forged article model (step S101). The forging analysis device 10 then calculates the distance between the forged article model and the die (step S102).

Thereafter, the forging analysis device 10 calculates the strain rate (step S103). The forging analysis device 10 then determines whether the forging process in which the calculation has been performed is a final process (step S104). If the forging analysis device 10 determines that the forging process is the final process (step S104: Yes), the forging analysis device 10 transmits the mesh data 14b, the distance data 14c, and the strain rate data 14d to the forging flaw determination device 20 (step S105), and ends the processing.

On the other hand, if the forging analysis device 10 determines that the process is not the final process (step S104: No), it reads out data of a forged article model for the next process (step S106), and proceeds to step S101.

### Processing Procedure of Forging Flaw Determination Device 20

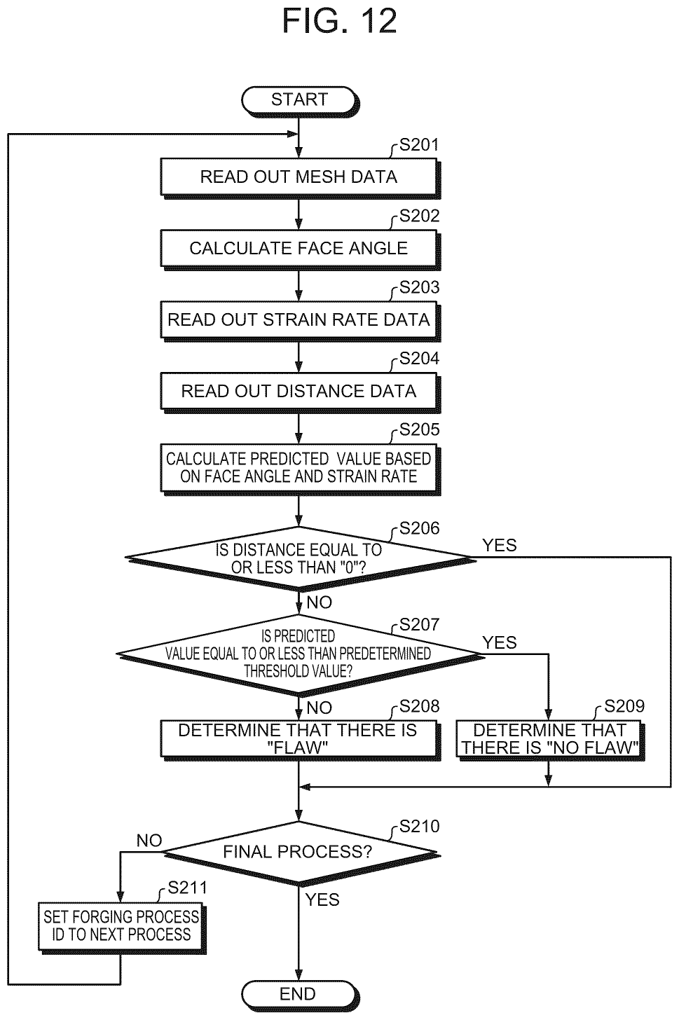

Next, a processing procedure of the forging flaw determination device 20 will be described. FIG. 12 is a flowchart showing the processing procedure of the forging flaw determination device 20 shown in FIG. 1B. As shown in FIG. 12, the forging flaw determination device 20 reads out the mesh data 24b from the storage unit 24 (step S201). The forging flaw determination device 20 then calculates the face angle (step S202).

Thereafter, the forging flaw determination device 20 reads out the strain rate data 24e from the storage unit 24 (step S203). Then, the forging flaw determination device 20 reads out the distance data 24d from the storage unit 24 (step S204). Thereafter, the forging flaw determination device 20 calculates a predicted value based on the face angle and the strain rate (step S205).

The forging flaw determination device 20 determines whether the distance equal to or less than “0” (Step S206). If the distance is equal to or less than “0” (Step S206: Yes), the forging flaw determination device 20 proceeds to Step S210.

On the other hand, if the distance is neither equal to nor less than “0” (step S206: No), the forging flaw determination device 20 determines whether the predicted value equal to or less than a predetermined threshold value (step S207). If the predicted value is equal to or less than the predetermined threshold value (step S207: Yes), the forging flaw determination device 20 determines that there is “no flaw” (step S209), and proceeds to step S210. If the predicted value is neither equal to nor less than the predetermined threshold value (step S207: No), the forging flaw determination device 20 determines that there is a “flaw” (step S208), and proceeds to step S210.

The forging flaw determination device 20 determines whether the forging process for which the determination has been performed is the final process (step S210). If the forging flaw determination device 20 determines that the forging process is the final process (step S210: Yes), the forging flaw determination device 20 ends the processing. On the other hand, if the forging flaw determination device 20 determines that the forging process is not the final process (step S210: No), the forging flaw determination device 20 sets the forging process ID to the next process (step S211), and proceeds to step S201.

### Display Example of Presence or Absence of Flaw

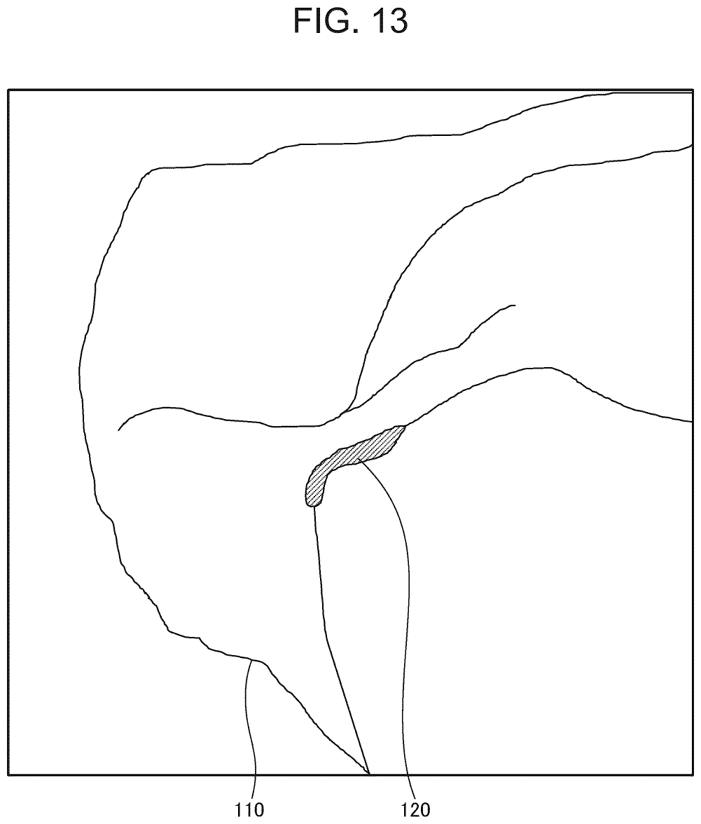

Next, a display example of the presence or absence of a flaw by the forging flaw determination device 20 will be described. FIG. 13 is a diagram showing a display example of the presence or absence of a flaw by the forging flaw determination device 20. As shown in FIG. 13, a location which has been determined as a flaw by the determination unit 25f is displayed as a flaw occurrence region 120 by the forging flaw determination device 20.

As described above, in the present embodiment, the forging flaw determination system includes the forging analysis device 10 and the forging flaw determination device 20. The forging analysis device 10 generates meshes for analyzing a forged article model, calculates the distance between the forged article model and the die, and calculates the strain rate in each analysis mesh. The forging flaw determination device 20 calculates the regression coefficient of the prediction formula for logistic regression analysis that calculates the predicted value, calculates the face angle between the mesh faces of adjacent meshes, calculates the predicted value using the prediction formula for logistic regression analysis based on the face angle and the strain rate, and determines the presence or absence of a flaw based on the predicted value and the distance.

In the present embodiment, description has been made on a case where the forging analysis device 10 performs generation of meshes, calculation of distances, and calculation of strain rates for all forging processes and transmits mesh data, distance data and strain rate data to the forging flaw determination device 20, and the forging flaw determination device 20 determines the presence or absence of a flaw based on the data received for all the forging processes. However, the forging analysis device 10 may perform generation of meshes, calculation of distances, and calculation of strain rates for each forging process, and transmits mesh data, distance data and strain rate data to the forging flaw determination device 20 each time the forging analysis device 10 performs generation of meshes, calculation of distances, and calculation of strain rates for each forging process, and the forging flaw determination device 20 may determine the presence or absence of a flaw based on data received for one forging process.

Furthermore, in the present embodiment, description has been made on a case where the forging flaw determination system includes the forging analysis device 10 for analyzing the forged article model in the forging process, and the forging flaw determination device 20 for determining the presence or absence of a flaw. However, the function of the forging analysis device 10 and the function of the forging flaw determination device 20 may be implemented by a single device.

Furthermore, in the present embodiment, description has been made on a case where the face angle and the strain rate are used as explanatory variables in the logistic regression analysis. However, other parameters affecting the occurrence of flaws in forging, such as the processing temperature, may be added as explanatory variables.

The configurations illustrated in the embodiments are merely functional schematics, and are not necessarily physically configured as illustrated. In other words, the form of distribution and integration of the respective devices are not limited to those illustrated in figures, and all or some of the devices can be configured to be functionally or physically distributed and integrated in any unit according to various loads, usage conditions, etc.

The forging flaw determination system, the forging flaw determination device, and the forging flaw determination method according to the present disclosure are suitable for a case where the presence or absence of a flaw is efficiently determined in forging.

What is claimed is:

1. A forging flaw determination system comprising:
a forging analysis device that is configured to generate a forged article model in a plurality of forging processes for forging and includes a first controller, the first controller being configured to calculate a strain rate based on an analysis mesh for the forged article model; and
a forging flaw determination device that is configured to predict, based on the forged article model, presence or absence of a flaw phenomenon when a forged article is formed in each forging process for the forging and includes a second controller, the second controller being configured to determine the presence or absence of the flaw phenomenon based on the strain rate and a face angle between faces of adjacent analysis meshes.

2. The forging flaw determination system according to claim 1, wherein the first controller is configured to:
generate the analysis mesh for the forged article model;
calculate a distance between the forged article model and a die; and
calculate the strain rate based on an amount of change in the forged article model between forging processes.

3. The forging flaw determination system according to claim 2, wherein the second controller is configured to:
calculate the face angle between the faces of the adjacent analysis meshes;
calculate a predicted value of the flaw phenomenon based on the strain rate and the face angle; and
determine the presence or absence of the flaw phenomenon based on the predicted value and the distance.

4. The forging flaw determination system according to claim 3, wherein the second controller is configured to calculate the predicted value of the flaw phenomenon by logistic regression analysis in which the strain rate and the face angle are set as explanatory variables and occurrence of the flaw phenomenon is set as an objective variable.

5. A forging flaw determination device that is configured to predict, based on a forged article model in a plurality of forging processes for forging, presence or absence of a flaw phenomenon when a forged article is formed in each forging process for the forging, the forging flaw determination device comprising a second controller that is configured to:
calculate a strain rate based on an analysis mesh for the forged article model; and
determine the presence or absence of the flaw phenomenon based on the strain rate and a face angle between faces of adjacent analysis meshes.

6. A forging flaw determination method to be executed in a forging flaw determination system, the forging flaw determination system including a forging analysis device configured to generate a forged article model in a plurality of forging processes for forging, and a forging flaw determination device configured to predict, based on the forged article model, presence or absence of a flaw phenomenon when a forged article is formed in each forging process for the forging, the forging flaw determination method comprising:
calculating, by the forging analysis device, a strain rate based on an analysis mesh for the forged article model; and
determining, the forging flaw determination device, the presence or absence of the flaw phenomenon based on the strain rate and a face angle between faces of adjacent analysis meshes.
